(19) United States
(12) Patent Application Publication
(10) Pub. No.: US 2022/0071544 A1
(43) Pub. Date: Mar. 10, 2022

(54) ELECTROENCEPHALOGRAM (EEG) SIGNAL AMPLIFICATION APPARATUS FOR BOOSTING IMPEDANCE

(71) Applicant: UNIST (ULSAN NATIONAL INSTITUTE OF SCIENCE AND TECHNOLOGY), Ulsan (KR)

(72) Inventors: Seong-Jin Kim, Ulsan (KR); Yong Jae Park, Ulsan (KR)

(73) Assignee: UNIST (ULSAN NATIONAL INSTITUTE OF SCIENCE AND TECHNOLOGY), Ulsan (KR)

(21) Appl. No.: 17/466,393

(22) Filed: Sep. 3, 2021

(30) Foreign Application Priority Data
Sep. 4, 2020 (KR) 10-2020-0113207

(51) Int. Cl. A61B 5/31; A61B 5/00; H03F 1/12

(52) U.S. Cl. CPC A61B 5/31; H03F 2200/378; H03F 1/12; A61B 5/4821

(57) ABSTRACT

There is provided an electroencephalogram (EEG) signal amplifier for boosting impedance in an analog front end (AFE). The EEG signal amplifier includes a first feedback loop configured to amplify an EEG signal, a second feedback loop connected to the first feedback loop and configured to amplify an input impedance, and an attenuator included in the second feedback loop.

### CROSS REFERENCE TO RELATED APPLICATION

This application claims priority under 35 U.S.C. § 119 to Korean Patent Application No. 10-2020-0113207, filed on Sep. 4, 2020, in the Korean Intellectual Property Office, the disclosure of which is incorporated by reference herein in its entirety.

### FIELD

The present disclosure relates to an electroencephalogram (EEG) signal amplification apparatus for boosting impedance in an analog front end (AFE).

Specifically, the present disclosure relates to a circuit for increasing the Input impedance of a measuring device when measuring an EEG signal for a long time to determine the depth of anesthesia of a patient during surgery and to prevent arousal. In addition, the present disclosure relates to a circuit for maximizing an impedance boosting effect in response to a change in a capacitor included in a DC servo loop (DSL).

### BACKGROUND

The number of deaths and arousal accidents during surgery is increasing due to incorrect anesthetic dose during medical surgery, and for this reason, it is very important to accurately determine the depth of anesthesia of a patient through EEG signal measurement during medical surgery.

Because the EEG signal is a very small signal, a medical device for measuring the EEG signal has to be implemented to generate very small noise. Meanwhile, due to the drying of a gel of an electrode during long-term surgery, a phenomenon in which the DC offset of a device is increased and a phenomenon in which the impedance of the electrode itself is increased may occur. Accordingly, the input impedance of the device should also be increased.

In conventional circuits, input impedance may be increased by implementing a small unit capacitor, but the small unit capacitor is difficult to implement accurately and has problems of non-linearity and mismatch, and thus, there is a need for improvement.

### SUMMARY

The present disclosure provides an electroencephalogram (EEG) signal amplification apparatus that cancels an Electrode DC Offset (EDO) while maintaining a high input impedance.

However, this goal is exemplary, and the scope of the present disclosure is not limited thereto.

Additional aspects will be set forth in part in the description which follows and, in part, will be apparent from the description, or may be learned by practice of the presented embodiments of the disclosure.

According to an embodiment of the present disclosure, there is provided an EEG signal amplifier including a first feedback loop configured to amplify an EEG signal, a second feedback loop connected to the first feedback loop and configured to amplify an input impedance, and an attenuator included in the second feedback loop.

The attenuator may have a gain greater than 0 and less than 1.

The EEG signal amplifier may further include a first DC Servo Loop (DSL) connected to the first feedback loop and the second feedback loop and configured to cancel an Electrode DC Offset (EDO), and a second DSL connected to the first DSL and configured to cancel an EDO based on a comparison result between an output voltage of the first DSL and a reference voltage.

The second DSL may include a single slope comparator configured to generate a flag signal and a count value signal as a result of comparing the output voltage with the reference voltage, and a capacitor operation portion configured to operate at least one capacitor among a plurality of capacitors based on the flag signal and the count value signal.

The second feedback loop may include a variable capacitor at an output terminal of the attenuator, and the EEG signal amplifier may further include a memory, wherein the memory may include information about a capacitance value of the variable capacitor corresponding to a change in a capacitance value of at least one of the plurality of capacitors.

The EEG signal amplifier is configured to control the capacitance value of the variable capacitor based on the information.

Additional aspects, features, and advantages other than described above will become apparent from the detailed description, claims, and drawings for implementing the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A shows an electroencephalogram (EEG) signal amplifier including a positive feedback loop;

FIG. 1B is a circuit diagram representing the EEG signal amplifier of FIG. 1A by re-modeling;

FIG. 2A shows a configuration of an EEG signal amplifier according to an embodiment of the present disclosure;

FIG. 2B is a circuit diagram representing the EEG signal amplifier of FIG. 2A by re-modeling;

FIG. 3 shows an EEG signal amplifier including a DC servo loop according to an embodiment of the present disclosure; and

FIG. 4 is a diagram illustrating a memory-based EEG signal amplifier according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, various embodiments of the present disclosure will be described with reference to the accompanying drawings. The present disclosure may have various modifications and embodiments and thus will be described in detail with respect to particular embodiments illustrated in the drawings. However, it should be understood that the embodiments are not intended to limit the scope of the present disclosure; rather, the present disclosure should be construed to cover all modifications, equivalents, and/or alternatives falling within the spirit and scope of the embodiments of the disclosure. In connection with descriptions of the drawings, like reference numerals denote like elements.

Throughout the present specification, the terms such as “include (comprise)” and/or “have” may be construed to indicate the presence of characteristics, numbers, steps, operations, components, elements, or combinations thereof, but may not be construed to exclude the presence or addition of one or more other characteristics, numbers, steps, operations, components, elements, or combinations thereof.

In the present disclosure, the term “or” includes any or all combinations of words enumerated together. For example, the expression “A or B” may include only A, only B, or both A and B.

The terms “first”, “second”, etc. used herein may modify various elements in various embodiments, but may not limit such elements. For example, these terms do not limit the order and/or importance of the elements, and may be used merely for distinguishing an element from another element.

Throughout the specification, it will be understood that when an element is referred to as being “connected” or “coupled” to another element, the element may be directly connected to or electrically coupled to the other element with one or more intervening elements interposed therebetween.

Terms such as “module”, “unit”, “part”, etc. used herein indicate an element for performing one or more functions or operations, and the element may be embodied as hardware or software or a combination of hardware and software. Furthermore, a plurality of “modules”, “units”, or “parts” may be integrated into at least one module or chip and implemented as at least one processor, except for a case where the respective “modules”, “units”, or “parts” need to be implemented as discrete particular hardware.

Terms such as those defined in a commonly used dictionary should be interpreted as having a meaning consistent with the context of a related art, and should not be construed in an ideal or overly formal sense unless explicitly defined in various embodiments of the present disclosure.

Hereinafter, various embodiments of the present disclosure will be described in detail with reference to the accompanying drawings.

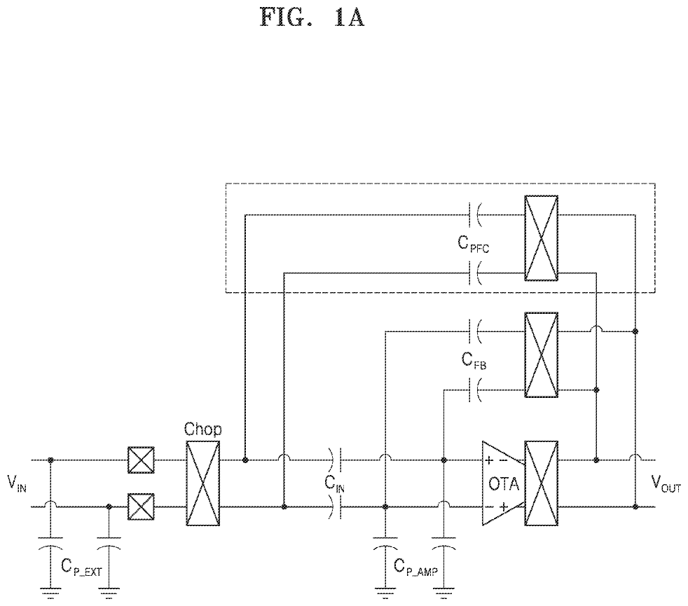

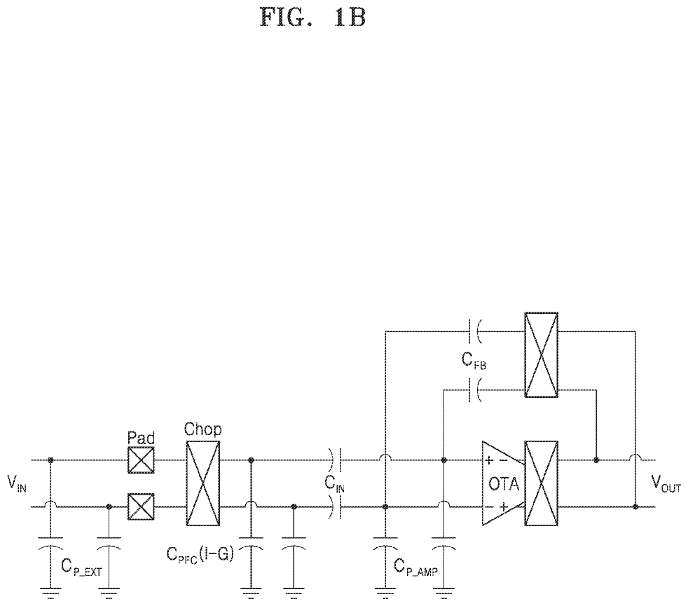

FIG. 1A shows an electroencephalogram (EEG) signal amplifier including a positive feedback loop.

The EEG signal amplifier may amplify an input EEG signal and output an amplified EEG signal. For example, the EEG signal amplifier may be an instrumentation amplifier (IA) that amplifies and outputs biological signals such as electrocardiogram (ECG), electromyography (EMG), and brainwave (i.e., EEG) in an analog front-end (AFE).

Because the EEG signal is a small signal, the EEG signal amplifier should also have small noise. In general cases other than depth of anesthesia, an Electrode DC Offset (EDO) is at a level of up to 50 mV. However, in the case of long-term surgery, as a gel of an electrode dries, the EDO may appear as large as about 200 mV to about 400 mV, which may be a problem.

The EEG signal amplifier according to an embodiment of the present disclosure may use a chopper generating small noise. Chopper control using a chopper is a control method of a circuit that modulates a DC or AC signal into the operating frequency band of the chopper by repeating ON/OFF of a current.

Meanwhile, input impedance Z_{IN} may be expressed as in Equation (1).

$${Z}_{IN} = \frac{1}{2 {F}_{C h o p} {C}_{I N}} \vert \vert \frac{1}{2 π {F}_{IN} {C}_{P - E X T}} \qquad [ Equation 1 ]$$

In this case, input impedance Z_{IN} is parallel combination of C_{IN} operated at chopping frequency F_{Chop} and C_{P-EXT} operated at input frequency F_{IN}. Although a structure using a chopper is common to have small noise, there is a disadvantage that the input impedance becomes as small as the chopper frequency when the chopper is used. That is, in general, in a structure using a chopper, the input impedance is inevitably reduced.

In order to solve this problem, the EEG signal amplifier of the present disclosure may include a positive feedback loop in a main path through which the EEG signal passes. That is, the EEG signal amplifier may improve input impedance by using a positive feedback loop including capacitor C_{PFC}. This will be further described with reference to FIG. 1B.

The main path may refer to a path connected to an EEG signal input terminal to which an input signal Vin is input and an EEG signal output terminal to which an output signal of an operation transconductance amplifier (OTA) is output.

Specifically, the positive feedback loop may be implemented as a closed loop including the capacitance C_{PFC} between an input portion of the OTA and an output portion of the OTA. The OTA may be an amplifier that outputs an applied input voltage as an output current in proportion to transconductance (Gm).

FIG. 1B is a circuit diagram representing the EEG signal amplifier of FIG. 1A by re-modeling.

A circuit including the positive feedback loop of FIG. 1A may be modeled as shown in FIG. 1B by using Miller Theorem. In particular, the capacitance C_{PFC} may be modeled equivalently as capacitance C_{PFC}(1−G). Referring to FIG. 1B, capacitor C_{P_EXT}, capacitor C_{PFC}(1−G), and capacitor C_{IN} are in parallel with each other, and the input impedance Z_{IN} is expressed by Equation (2).

$${Z}_{IN} = \frac{1}{2 {F}_{C h o p} ( {C}_{I N} + {C}_{PFC} ( 1 - G ) )} \vert \vert \frac{1}{2 π {F}_{IN} {C}_{P - E X T}} \qquad [ Equation 2 ]$$

In this case, G is a gain formed based on the capacitance C_{IN} and capacitance C_{FB}. When G is positive, the capacitance C_{PFC}(1−G) becomes negative. The capacitance C_{PFC}(1−G) may cancel the capacitance C_{IN} connected in parallel thereto, which has the effect that the capacitance C_{IN} itself becomes smaller in Equation 2, thereby increasing the input impedance Z_{IN}.

Meanwhile, in order to maintain the stability of the EEG signal amplifier, the condition of Equation 3 has to be satisfied.

$${C}_{I N} + {C}_{P - E X T} \cdot \frac{π {F}_{IN}}{{F}_{C h o p}} > {C}_{P F C} ( G - 1 ) \qquad [ Equation 3 ]$$

The input impedance Z_{IN} increases as the value of the capacitance C_{PFC} is closer to the value of

$$( {C}_{I N} + {C}_{P - E X T} \cdot \frac{π {F}_{IN}}{{F}_{C h o p}} ) \text{/} ( G - 1 ) .$$

However, when the value of the capacitance C_{PFC} is equal to the value of

$$( {C}_{I N} + {C}_{P - E X T} \cdot \frac{π {F}_{IN}}{{F}_{C h o p}} ) \text{/} ( G - 1 ) ,$$

the input impedance Z_{IN} diverges to infinity. That is, by reducing the unit capacitance of the capacitance C_{PFC}, the input impedance Z_{IN} may be increased, but it is difficult to accurately implement the unit capacitance that satisfies the stability of the input impedance Z_{IN}, and there may be problems of non-linearity and mismatch.

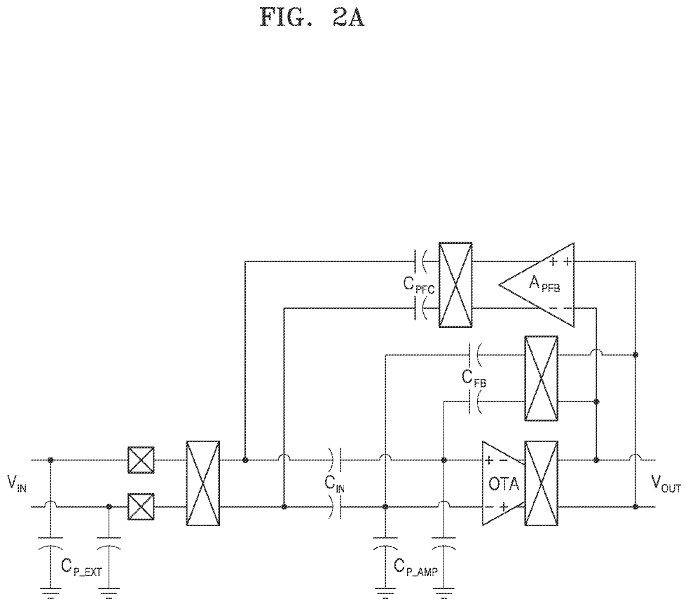

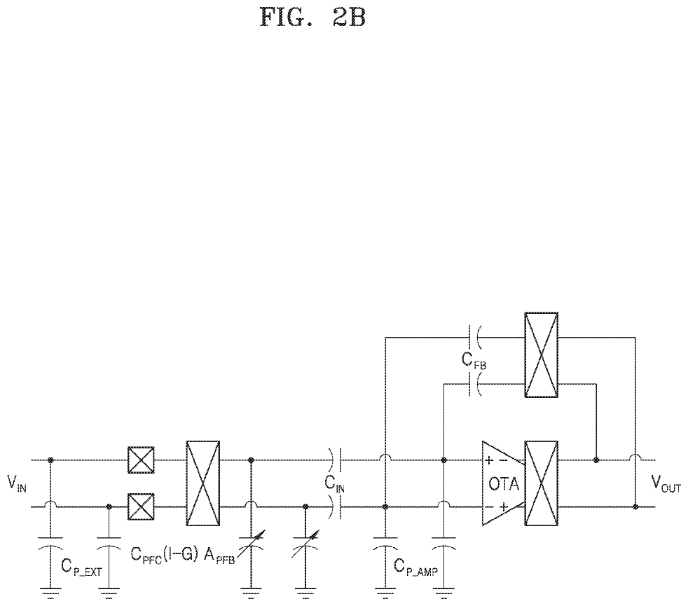

FIG. 2A shows a configuration of an EEG signal amplifier according to an embodiment of the present disclosure.

Referring to FIG. 2A, the EEG signal amplifier according to the embodiment of the present disclosure may include a positive feedback loop including an attenuator A_{PFB} added to FIG. 1A.

The attenuator A_{PFB} is included in the positive feedback loop, and may be installed to form a closed loop between the output terminal of an OTA and capacitance C_{PFC}. In this case, the gain of the attenuator A_{PFB} may be greater than 0 and less than 1.

FIG. 2B is a circuit diagram representing the EEG signal amplifier of FIG. 2A by modeling.

The circuit of FIG. 2A may be modeled as shown in FIG. 2B by using Miller Theorem. In particular, a closed loop circuit including the capacitance C_{PFC} and the attenuator A_{PFB} may be modeled equivalently as capacitance C_{PFC}(1−G)A_{PFB}.

Referring to FIG. 2B, capacitance C_{P_EXT}, capacitance C_{PFC}(1−G)A_{PFB}, and capacitance C_{IN} are in parallel with each other, and the input impedance Z_{IN} is expressed by Equation (4).

$${Z}_{IN} = \frac{1}{2 {F}_{C h o p} ( {C}_{I N} + {C}_{PFC} ( 1 - G ) {A}_{PFB} )} \vert \vert \frac{1}{2 π {F}_{IN} {C}_{P - E X T}} \qquad [ Equation 4 ]$$

Meanwhile, in order to maintain the stability of the EEG signal amplifier, the condition of Equation 5 has to be satisfied.

$${C}_{I N} + {C}_{P - E X T} \cdot \frac{π {F}_{IN}}{{F}_{C h o p}} > {C}_{P F C} ( G - 1 ) {A}_{P F B} \qquad [ Equation 5 ]$$

Even though the unit capacitance of the capacitance C_{PFC} is large, since the value of the attenuator A_{PFB} is less than 1, there may be an effect of reducing the unit capacitance of the capacitance C_{PFC}. For example, assuming that the circuits according to FIGS. 2A and 2B is implemented with the attenuator A_{PFB} having a value of 0.1, even though the unit capacitance of the capacitance C_{PFC} is about 10 times greater, the input impedance of the same size may be maintained.

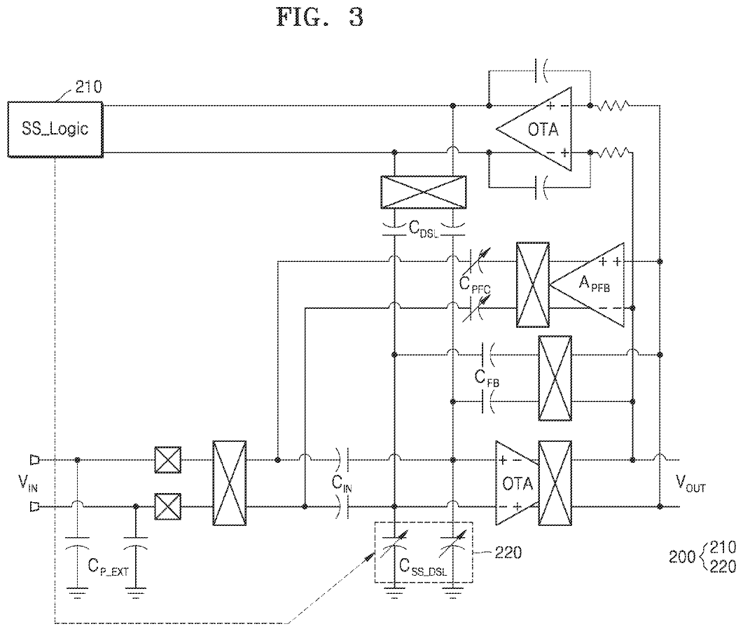

FIG. 3 shows an EEG signal amplifier including a DC servo loop according to an embodiment of the present disclosure.

Referring to FIG. 3, the EEG signal amplifier may include an Analog DC Servo Loop (ADSL) 200 according to an embodiment of the present disclosure.

In addition, the EEG signal amplifier may include a digital DC servo loop for processing an increase in EDO due to long-term surgery. The digital DC servo loop may include a single slope comparator 210 and a capacitor operation portion 220.

A DC servo loop is a negative feedback loop using an amplifier (e.g. operational amplifier (OP-AMP), OTA, etc.) having high DC gain and relatively low high-frequency gain, and may filter high-frequency current and amplify only low-frequency current.

In the EEG signal amplifier according to the embodiment of the present disclosure, the ADSL 200 may form a closed loop with a main path and a negative feedback, thereby canceling an EDO included in the EEG signal.

Specifically, when using a chopper, there is a problem that a DC signal including an EDO is modulated and is amplified together with the EEG signal. An EDO cancellation circuit in FIG. 3 for solving this problem may filter only the DC signal at an output (V_{OUT}) node through negative feedback having the ADSL 200.

However, in order to cancel a large EDO in the above-described method, capacitance C_{DSL} inside the ADSL 200 increases, and thus, the noise of the EEG signal amplifier is inevitably increased. In other words, to increase EDO cancellation range, the capacitance C_{DSL} has to be increased, but when the capacitance C_{DSL} is increased, noise increases. Accordingly, there is a limit in increasing the value of the capacitance C_{DSL}.

The single slope comparator 210 may be connected to an output terminal of an OTA included in the ADSL 200. The single-slope comparator 210 may use a single-slope algorithm to cancel a large amount of EDO while accompanying a small noise.

In this case, the single slope comparator 210 may include a comparator (not shown) for comparing a reference voltage with an output voltage from the ADSL 200 in response to a preset comparison period, and a controller (not shown) for generating a flag signal and a count value signal based on a comparison result by the comparator. Each of the comparator and the controller may be implemented as a separate chip, but is not limited thereto.

Specifically, the comparator 210 may sense a voltage of the output terminal of the ADSL 200, and when the voltage of the output terminal is higher than a preset reference voltage, the controller may generate a flag signal and a count value signal for operating the capacitor operation portion 220. In this case, the flag signal and the count value signal may be digital signals.

The capacitor operation portion 220 may turn on at least one capacitor among a plurality of capacitors C_{SS_DSL} in response to the flag signal and/or the count value signal from the single slope comparator 210, and may be connected to an input terminal of the OTA in the main path through the turned-on capacitor to form a negative feedback for closed loop connection.

Specifically, when the flag signal is 0, the capacitor operation portion 220 may maintain a capacitor C_{SS_DSL} that is previously turned on, and when the flag signal is 1, the capacitor operation portion 220 may control the plurality of capacitors C_{SS_DSL} to be turned on by a number corresponding to the count value signal.

For example, it is assumed that the ADSL 200 may track an EDO of about 50 mV and each of the plurality of capacitors C_{SS_DSL} may cancel an EDO of about 25 mV. In this case, when all of the capacitors (e.g., 15 capacitors) included in the capacitor operation portion 220 are turned on, the digital DC servo loop may cancel the total EDO of about 375 mV. That is, the EEG signal amplifier may cancel the total EDO of about 425 mV.

By using the single slope comparator 210 and the capacitor operation portion 220, the EEG signal amplifier according to the embodiment of the present disclosure may cancel a significantly increased EDO compared to when only the ADSL 200 is used.

In the case of the embodiment shown in FIG. 3, when the capacitance value of each of the capacitors C_{SS_DSL} increases, the capacitance C_{PFC} also increases accordingly. When the capacitance C_{PFC} increases, there is a risk that an input impedance higher than an intended input impedance Z_{IN} may be formed or an unstable input impedance may be formed.

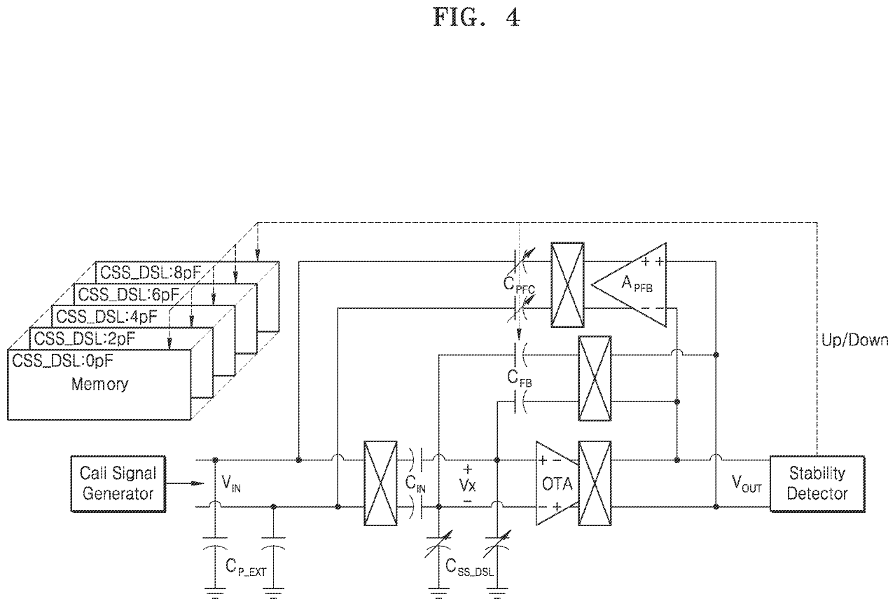

FIG. 4 is a diagram illustrating a memory-based EEG signal amplifier according to an embodiment of the present disclosure.

In a positive feedback loop of the EEG signal amplifier according to an embodiment of the present disclosure, a voltage Vx in FIG. 4 is not in an AC ground state, that is, a state in which an AC component is 0, due to the influence of a chopper. Also, because an additional capacitor C_{SS_DSL} is added to the Vx node to cancel an EDO, the additional capacitor C_{SS_DSL} may affect the capacitance C_{PFC}.

When the capacitance value of the capacitor C_{SS_DSL} increases due to an increase in EDO, the capacitance C_{PFC} also increases as described above. The EEG signal amplifier according to an embodiment of the present disclosure may prevent a side effect that, as the capacitance C_{PFC} increases, an input impedance higher than the intended input impedance Z_{IN} is formed or an unstable input impedance is formed.

Specifically, the EEG signal amplifier according to an embodiment of the present disclosure may include a memory, and the memory may store a calibrated value of the capacitance C_{PFC} for a changing capacitance value of each capacitor C_{SS_DSL}.

A calibration signal generator in FIG. 4 may provide a test signal. A stability detector in FIG. 4 may identify the capacitance value of the capacitor C_{SS_DSL}, which changes to remove the EDO in response to the test signal. In addition, the stability detector may obtain a capacitance C_{PFC} value that provides a high input impedance while maintaining stability, in response to a changing capacitance value of the capacitor C_{SS_DSL}.

The memory may store information obtained by the stability detector. That is, the memory may store information about the capacitance value of the capacitor C_{SS_DSL}, which changes in response to an EEG signal, and information about the capacitance C_{PFC} value that changes in response to the changing capacitance value of the capacitor C_{SS_DSL}.

In other words, the EEG signal amplifier according to an embodiment of the present disclosure may flexibly change the capacitance value of the capacitor C_{SS_DSL} in response to a changing EEG signal value, and thus may increase an EDO cancellation amount without increasing the capacitance value of the capacitor C_{DSL} included in the ADSL 200 by more than a threshold by using a sequentially increasing single-slope signal.

In addition, the EEG signal amplifier according to an embodiment of the present disclosure may stably control the input impedance by changing the capacitance C_{PFC} value in response to the capacitance value of the capacitor C_{SS_DSL}, which is flexibly changed based on a value stored in the memory.

The EEG signal amplifier according to an embodiment of the present disclosure may have an effect of easily boosting the input impedance Z_{IN} by including an attenuator for unit capacitance required in a positive feedback loop, and may also have an effect of flexibly adjusting the capacitance C_{PFC} value according to the change of external capacitance C_{P-EXT} and the change of the capacitance value of the capacitor C_{SS_DSL} for EDO processing.

In addition, embodiments of the disclosure described above may be implemented as a software program including instructions stored in a recording medium that is readable by a computer or a similar device using software, hardware, or a combination thereof. In some cases, the embodiments described herein may be implemented as the processor itself. According to the software implementation, embodiments such as procedures and functions described in the present specification may be implemented as separate software modules. Each of the software modules may perform one or more functions and operations described herein.

A recording medium that may be readable by a device may be provided in the form of a non-transitory computer-readable recording medium. In this regard, the term ‘non-transitory’ only means that the recording medium does not include a signal and is tangible, and the term does not distinguish between data that is semi-permanently stored and data that is temporarily stored in the recording medium. The non-transitory computer-readable recording medium refers to a medium that stores data semi-permanently and is readable by a device and not a medium storing data for a short time, such as a register, a cache, a memory, etc. Examples of the non-transitory computer-readable recording medium may include a CD, a digital versatile disk (DVD), a hard disk, a Blu-ray disk, a USB, a memory card, ROM, etc.

The EEG signal amplifier according to an embodiment of the present disclosure may have an effect of easily boosting input impedance by including an attenuator for unit capacitance required in a positive feedback loop.

In addition, the EEG signal amplifier according to the embodiment of the present disclosure may have an effect of stably controlling input impedance by flexibly adjusting the capacitance value of a positive feedback loop according to the change of external capacitance and the change of capacitance for EDO processing.

Despite these effects, the scope of the disclosure is not limited thereby.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

We claim:

1. An electroencephalogram (EEG) signal amplifier comprising:
a first feedback loop configured to amplify an EEG signal;
a second feedback loop connected to the first feedback loop and configured to amplify an input impedance; and
an attenuator included in the second feedback loop.

2. The EEG signal amplifier of claim 1, wherein the attenuator has a gain greater than 0 and less than 1.

3. The EEG signal amplifier of claim 1, further comprising:
a first DC Servo Loop (DSL) connected to the first feedback loop and the second feedback loop and configured to cancel an Electrode DC Offset (EDO); and
a second DSL connected to the first DSL and configured to cancel an EDO based on a comparison result between an output voltage of the first DSL and a reference voltage.

4. The EEG signal amplifier of claim 3, wherein the second DSL includes:
a single slope comparator configured to generate a flag signal and a count value signal as a result of comparing the output voltage with the reference voltage; and
a capacitor operation portion configured to operate at least one capacitor among a plurality of capacitors based on the flag signal and the count value signal.

5. The EEG signal amplifier of claim 4, wherein
the second feedback loop includes a variable capacitor at an output terminal of the attenuator,
the EEG signal amplifier further comprising:
a memory,
wherein the memory includes information about a capacitance value of the variable capacitor corresponding to a change in a capacitance value of at least one of the plurality of capacitors.

6. The EEG signal amplifier of claim 5, wherein the EEG signal amplifier is configured to control the capacitance value of the variable capacitor based on the information.
